(19) United States
(12) Patent Application Publication
(10) Pub. No.: US 2023/0098394 A1
(43) Pub. Date: Mar. 30, 2023

(54) PRODUCTION OF LACTIC ACID FROM ORGANIC WASTE USING COMPOSITIONS OF BACILLUS COAGULANS SPORES

(71) Applicant: TRIPLE W LTD., Netanya (IL)

(72) Inventors: Ofir Avidan, Lod (IL); Tsvika Greener, Ness Ziona (IL)

(21) Appl. No.: 17/909,983

(22) PCT Filed: Mar. 24, 2021
(86) PCT No.: PCT/IL2021/050327

§ 371 Date: Sep. 7, 2022

Related U.S. Application Data
(60) Provisional application No. 62/993,759, filed on Mar. 24, 2020.

(51) Int. Cl. C12P 7/56; C12M 1/40; C12N 1/20; C12N 9/26; C12N 9/42; C12N 9/24; C12N 3/00

(52) U.S. Cl. CPC C12P 7/56; C12M 21/18; C12N 1/20; C12N 9/2411; C12N 9/2437; C12N 9/248; C12N 3/00; C12R 2001/07

(57) ABSTRACT

Systems and methods for recycling of organic waste to produce lactic acid by fermentation are provided, which utilize dried or partially-dried compositions of spores of the lactic acid-producing bacterium Bacillus coagulans.

### FIELD OF THE INVENTION

The present invention relates to industrial recycling of organic waste to produce lactic acid by fermentation processes, which utilize dried or partially-dried compositions of spores of the lactic acid-producing bacterium Bacillus coagulans.

### BACKGROUND OF THE INVENTION

Lactic acid fermentation, namely, production of lactic acid from carbohydrate sources via microbial fermentation, has been gaining interest in recent years due to the ability to use lactic acid as a building block in the manufacture of bioplastics. Lactic acid can be polymerized to form the biodegradable and recyclable polyester polylactic acid (PLA), which is considered a potential substitute for plastics manufactured from petroleum. PLA is used in the manufacture of various products including food packaging, disposables, fibers in the textile and hygiene products industries, and more. PLA is the most widely used plastic filament material in 3D printing.

Production of lactic acid by fermentation bioprocesses is preferred over chemical synthesis methods for various considerations, including environmental concerns, costs and the difficulty to generate enantiomerically pure lactic acid by chemical synthesis, which is desired for most industrial applications of PLA. The conventional fermentation process is typically based on anaerobic fermentation by lactic acid-producing microorganisms, which produce lactic acid as the major metabolic end product of carbohydrate fermentation. For production of PLA, the lactic acid generated during the fermentation is separated from the fermentation broth and purified by various downstream processes, and the purified lactic acid is then subjected to polymerization.

Lactic acid has a chiral carbon atom and therefore exists in two enantiomeric forms, D- and L-lactic acid. In order to generate PLA that is suitable for industrial applications, the polymerization process should utilize only one enantiomer. Presence of impurities or a racemic mixture of D- and L-lactic acid results in a polymer having undesired characteristics such as low crystallinity and low melting temperature. Thus, lactic acid bacteria that produce only L-lactate enantiomer or only D-lactate enantiomer are typically used.

In currently available commercial processes, the carbohydrate source for lactic acid fermentation is typically a starch-containing renewable source such as corn and cassava root. Additional sources, such as the cellulose-rich sugarcane bagasse, have also been proposed.

An additional source of carbohydrates for lactic acid fermentation that has been proposed is complex organic waste, such as mixed food waste from municipal, industrial and commercial origin. Such organic waste is advantageous as it is readily available and less expensive compared to other carbohydrate sources for lactic acid fermentation. However, the conversion of complex organic wastes to useful fermentation products such as lactic acid on an industrial scale faces numerous technical challenges and requires precise control over operational conditions, including pretreatment, pH, temperature, microbes and more. Improvements are needed in order to make the process economically feasible on an industrial scale.

Rosenberg et al. (2005) Biotechnology Letters, 27: 1943-1947 report the immobilization of Bacillus coagulans spores in polyvinylalcohol (PVA) hydrogel, lens-shaped capsules known as LentiKats®, and use of the immobilized spores in a lactic acid production from glucose.

EP 1504109 discloses a method for the production of lactic acid or a salt thereof wherein starch is subjected to a process of simultaneous saccharification and fermentation, the method comprising saccharifying starch in a medium comprising at least a glucoamylase, and in case the starch is in solid form a liquefaction step, and simultaneously fermenting the starch using a microorganism, and optionally isolating lactic acid from the medium, characterized in that a moderately thermophilic lactic acid-producing microorganism is used, which is adapted to the pH range of 5-5.80 and wherein said microorganism is derived from a strain of Bacillus coagulans, Bacillus thermoamylovorans, Bacillus smithii, Geobacillus stearothermophilus or from a mixture thereof.

EP 3174988 discloses a method for preparing a fermentation product comprising lactic acid, said method comprising: a) treating lignocellulosic material with caustic magnesium salt in the presence of water to provide treated aqueous lignocellulosic material; b) saccharifying the treated aqueous lignocellulosic material in the presence of a hydrolytic enzyme to provide a saccharified aqueous lignocellulosic material comprising fermentable carbohydrate and a solid lignocellulosic fraction; c) simultaneously with step b), fermenting the saccharified aqueous lignocellulosic material in the presence of both a lactic acid forming microorganism and caustic magnesium salt to provide an aqueous fermentation broth comprising magnesium lactate and a solid lignocellulosic fraction; d) recovering magnesium lactate from said broth, wherein said saccharification and said fermentation are carried out simultaneously.

WO 2008/043368 discloses a method of producing endospores of thermophilic sporogenic microbial strains, for example, Bacillus coagulans SIM7 DSM 14043, and the use thereof for inoculation of fermentation processes.

WO 2018/163094 discloses methods for inducing sporulation in Bacillus coagulans strains for use as probiotics, wherein excessive sporulation is induced by presence of certain nutrients and minerals up to a level of 10⁹ spores/ml.

WO 2017/122197, assigned to the Applicant of the present invention, discloses dual action lactic-acid (LA)-utilizing bacteria genetically modified to secrete polysaccharide-degrading enzymes such as cellulases, hemicellulases, and amylases, useful for processing organic waste both to eliminate lactic acid present in the waste and degrade complex polysaccharides.

There remains a need to improve the production of lactic acid from organic waste on an industrial scale, in order to make the process more economically feasible. It would be highly advantageous to have systems and methods that simplify the process, reduce costs and improve the overall yield.

### SUMMARY OF THE INVENTION

The present invention provides systems and methods for recycling organic waste to produce lactic acid on an industrial scale, utilizing dried or partially-dried compositions of Bacillus coagulans spores. The systems and methods of the present invention enable producing lactic acid on-site at organic waste management facilities without the need for complicated seed lines and controlled conditions for growing the cells prior to inoculation of the production fermenter.

The present invention further provides dried compositions of B. coagulans spores that are ready for inoculation into lactic acid production fermenters without a need for any activation or conditioning, optionally in combination with saccharide-degrading enzyme(s). The compositions disclosed herein comprise the spores formulated with magnesium lactate, and are characterized by prolonged stability of the spores at room temperature.

According to some embodiments, the dried compositions are suspended in a magnesium hydroxide slurry prior to inoculation of the spores to the lactic acid production fermenter. It was surprisingly found by the inventors of the present invention that the spores survive suspension in a magnesium hydroxide slurry and successfully germinate following such treatment. The present invention therefore provides simple means for inactivating microbial contaminants that may be present in the dried composition, prior to inoculation into the production fermenter.

According to particular embodiments, the present invention is directed to production of lactic acid from mixed food waste, municipal waste and agricultural waste. As disclosed herein, a dried or partially-dried (semi-dried) composition of Bacillus coagulans spores is inoculated into a lactic acid-production fermenter with pretreated organic waste that was subjected to pretreatment comprising reduction of particle size and optionally sterilization. As disclosed herein, the spores of Bacillus coagulans from the dried or partially-dried inoculum successfully germinate in the fermenter in the presence of organic waste from various sources, and ferment the organic waste to produce lactic acid at high yields.

The present invention advantageously allows simple integration of lactic acid production into organic waste management facilities, for on-site production of lactic acid from the organic waste. Conventionally, industrial fermentation processes involve seed lines, also termed seed trains, where banked cell samples are expanded to finally provide sufficient biomass to inoculate the main fermenter. A conventional seed train process begins with thawing of a cryopreserved cell bank vial, followed by multiple successive propagations into progressively larger culture vessels. When culture volume and cell density meet predetermined criteria, the culture is transferred to a production bioreactor in which cells continue to grow and divide and produce the desired product. Conventional seed train processes are time-consuming due to the number of culturing steps, and due to the low cell numbers in the cryopreserved cell-bank vial. In addition, sterility is required for inoculating each culture vessel, including the main production fermenter.

The present invention avoids the need for seed lines at the production site and provides simple means for sterile inoculation, thus saving both capital expenditure (CAPEX) and operational expenditure (OPEX). Compositions of dried or partially-dried spores as disclosed herein can be easily transported to organic waste management sites, stored and removed from storage upon need. It was surprisingly found that the spores in the dried or partially-dried compositions can successfully recover from storage, germinate and ferment organic waste to lactic acid at high yields. Advantageously, the dried or partially-dried compositions of spores do not require cooling and sustain various storage conditions for prolonged periods of time. As exemplified hereinbelow, viability of the spores is maintained throughout storage, and cell loss following drying and storage is minimal.

The utilization of organic waste as a substrate for fermentation as described herein is highly advantageous compared to previously described lactic acid production processes which utilize source materials that are of high value as human food.

As further disclosed herein, the dried or semi-dried compositions of spores can be inoculated into the fermenter together with a saccharide-degrading enzyme to obtain simultaneous saccharification and fermentation. Remarkably, no or minimal lag time is observed until lactic acid is produced.

According to one aspect, the present invention provides a method for recycling organic waste to produce lactic acid or a salt thereof, the method comprising:

(i) providing a pretreated organic waste that was subjected to pretreatment comprising reduction of particle size and optionally sterilization;

(ii) providing a dried composition of Bacillus coagulans spores;

(iii) mixing the pretreated organic waste in a fermentation reactor with one or more saccharide-degrading enzyme and the dried composition of B. coagulans spores, and incubating the mixture in the fermentation reactor to saccharify the organic waste and induce germination of the spores and subsequently lactic acid production by vegetative B. coagulans cells that germinate from the spores; and

(iv) recovering lactic acid or a salt thereof from the fermentation broth.

In some embodiments, the method further comprises suspending the dried composition of B. coagulans spores in a magnesium hydroxide slurry prior to the mixing with the pretreated organic waste in step (iii), thereby obtaining a B. coagulans spore suspension in which microbial contaminants are inactivated. In some embodiments, the concentration of magnesium hydroxide in the slurry is in the range of 1%-25%. In additional embodiments, the concentration of magnesium hydroxide in the slurry is in the range of 10%-20%. In yet additional embodiments, the concentration of magnesium hydroxide in the slurry is in the range of 5%-25%. Exemplary concentrations of magnesium hydroxide include 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%. Each possibility represents separate embodiment.

Suspension in magnesium hydroxide may be carried out for a few minutes up to several hours. Preferably, the suspending in a magnesium hydroxide slurry comprises incubating the suspension for between 15 minutes to 3 hours at a temperature between 25-60° C., preferably at a temperature between 50-60° C. In some embodiments, the suspending in a magnesium hydroxide slurry comprises incubating the suspension for 15-90 min at a temperature between 25-60° C. In additional embodiments, the suspending in a magnesium hydroxide slurry comprises incubating the suspension for 15-90 min at a temperature between 50-55° C. In some embodiments, the suspending in a magnesium hydroxide slurry comprises incubating the suspension for 30-90 min or 30-60 min at a temperature between 25-60° C. Each possibility represents a separate embodiment. In additional embodiments, the suspending in a magnesium hydroxide slurry comprises incubating the suspension for 30-90 min or 30-60 min at a temperature between 50-55° C. In some embodiments, suspension in a magnesium hydroxide slurry is carried out at room temperature.

In some embodiments, the dried composition of B. coagulans spores comprises magnesium lactate.

In some embodiments, the organic waste is selected from the group consisting of food waste, municipal waste, agricultural waste, plant material and a mixture or combination thereof.

In some embodiments, the incubating is carried out at a pH in the range of 5-7. In some particular embodiments, the incubating is carried out at a pH in the range of 5.5-6.5.

In some embodiments, the incubating is carried out at a temperature in the range of 45-60° C. In some particular embodiments, the incubating is carried out at a temperature in the range of 50-55° C.

In some embodiments, the incubating in step (iii) is carried out for a period of time in the range of 20-48 hours. In some particular embodiments, the incubating in step (iii) is carried out for a period of time in the range of 20-36 hours.

In some embodiments, the one or more saccharide-degrading enzyme is a polysaccharide-degrading enzyme selected from the group consisting of an amylase, a cellulase and a hemicellulose.

In some embodiments, the one or more saccharide-degrading enzyme comprises a glucoamylase.

In some embodiments, the mixing in step (iii) comprises adding the dried composition of B. coagulans to the fermentation reactor to obtain at least 10\hat{ }4 spores/ml fermentation medium. In additional embodiments, the mixing in step (iii) comprises adding the dried composition of B. coagulans to the fermentation reactor to obtain at least 10\hat{ }6 spores/ml fermentation medium.

A dried inoculum of spores as disclosed is characterized by moisture content of up to 15% (w/w) or any amount therebetween. In some embodiments, the dried composition of B. coagulans spores is characterized by moisture content of up to 10% (w/w). In some embodiments, the dried composition of B. coagulans spores is characterized by moisture content of 4%-15% (w/w), for example 4%-10% (w/w). Each possibility represents a separate embodiment of the present invention.

As provided herein, the moisture content of a dried or semi-dried inoculum, formulation or composition comprising B. coagulans spores refers to the amount of water outside the spores (namely, “moisture content” as used herein does not include water found inside the spores). The moisture content is provided as a percentage out of the total weight of the inoculum, formulation or composition. The terms “inoculum”, “formulation” and “composition” of spores are used herein interchangeably to describe a composition containing the spores, wherein the composition may be dried or semi-dried.

According to a further aspect, the present invention provides a system for recycling organic waste to produce lactic acid or a salt thereof, the system comprising:

(a) a source of pretreated organic waste that was subjected to pretreatment comprising reduction of particle size and optionally sterilization;

(b) a dried composition of Bacillus coagulans spores;

(c) one or more saccharide-degrading enzyme; and

(d) a fermentation reactor for mixing therein the pretreated organic waste, the one or more saccharide-degrading enzyme and the dried composition of B. coagulans spores,
wherein the mixture is incubated in the fermentation reactor to saccharify the organic waste and induce germination of the spores and subsequently lactic acid production by vegetative B. coagulans cells that germinate from the spores.

In some embodiments, the system comprises:
(a) a source of pretreated organic waste that was subjected to pretreatment comprising reduction of particle size and optionally sterilization;
(b) a dried composition of B. coagulans spores suspended in a magnesium hydroxide slurry;
(c) one or more saccharide-degrading enzyme; and
(d) a fermentation reactor for mixing therein the pretreated organic waste, the one or more saccharide-degrading enzyme and the dried composition of B. coagulans spores suspended in a magnesium hydroxide slurry,
wherein the mixture is incubated in the fermentation reactor to saccharify the organic waste and induce germination of the spores and subsequently lactic acid production by vegetative B. coagulans cells that germinate from the spores.

According to a further aspect, the present invention provides a dried inoculum in a powder form for lactic acid fermentation, comprising spores of Bacillus coagulans; and magnesium lactate, wherein the inoculum is dried and ready for inoculation into a lactic acid production fermenter to provide lactic acid production.

In some embodiments, the dried inoculum comprises 10\hat{ }8-10\hat{ }10 spores/g powder, and the concentration of the magnesium lactate in the dried inoculum is in the range of 40-60% (w/w).

In some embodiments, a method for recycling organic waste to produce lactic acid or a salt thereof is provided, the method comprising:

(i) providing the dried inoculum comprising B. coagulans spores and magnesium lactate;

(ii) suspending the dried inoculum in a magnesium hydroxide slurry, thereby obtaining a B. coagulans spore suspension in which microbial contaminants are inactivated;

(iii) mixing the suspension obtained in step (ii) in a fermentation reactor with one or more saccharide-degrading enzyme and pretreated organic waste that was subjected to pretreatment comprising reduction of particle size and optionally sterilization, and incubating to saccharify the organic waste and induce germination of the spores and subsequently lactic acid production by vegetative B. coagulans cells that germinate from the spores; and

iv) recovering lactic acid or a salt thereof from the fermentation broth.

The methods disclosed herein are particularly beneficial for the production of magnesium lactate. In some embodiments, the method is a method for producing magnesium lactate. In some embodiments, a method for recycling organic waste to produce magnesium lactate is provided, the method comprising:

providing a pretreated organic waste that was subjected to pretreatment comprising reduction of particle size and optionally sterilization;

providing a dried composition of B. coagulans spores, comprising B. coagulans and magnesium lactate;

suspending the dried composition of B. coagulans spores in a magnesium hydroxide slurry, thereby obtaining a B. coagulans spore suspension in which microbial contaminants are inactivated;

mixing the pretreated organic waste in a fermentation reactor with one or more saccharide-degrading enzyme and the B. coagulans spore suspension;

incubating the mixture in the fermentation reactor to saccharify the organic waste and induce germination of the spores and subsequently lactic acid production by vegetative B. coagulans cells that germinate from the spores, wherein an alkaline compound selected from magnesium hydroxide, magnesium oxide and magnesium carbonate is added to the fermentation reactor during the incubation to adjust pH, thereby obtaining lactate monomers and Mg²⁺ ions; and
recovering magnesium lactate from the fermentation broth.

In some particular embodiments, the alkaline compound added to the fermentation reactor during the incubation to adjust pH is magnesium hydroxide.

According to a further aspect, there is provided a method for recycling organic waste to produce lactic acid or a salt thereof, the method comprising:

(i) providing a pretreated organic waste that was subjected to pretreatment comprising reduction of particle size and optionally sterilization;

(ii) providing a partially-dried composition of Bacillus coagulans spores, characterized by a moisture content in the range of 15%-30% (w/w);

(iii) mixing the pretreated organic waste in a fermentation reactor with one or more saccharide-degrading enzyme and the partially-dried composition of B. coagulans spores, and incubating the mixture in the fermentation reactor to saccharify the organic waste and induce germination of the spores and subsequently lactic acid production by vegetative B. coagulans cells that germinate from the spores; and

(iv) recovering lactic acid or a salt thereof from the fermentation broth.

In some embodiments, the partially-dried composition of B. coagulans spores is characterized by a moisture content in the range of 15%-25% (w/w).

Other objects, features and advantages of the present invention will become clear from the following description and examples.

### BRIEF DESCRIPTION OF THE FIGURES

FIG. 1. Inhibition of live microbial cells by Mg(OH)₂. Escherichia coli BL21, Bacillus subtilis strain 169 and Saccharomyces cerevisiae were incubated in LB (A) or 15% Mg(OH)₂ (B) at 52° C. for 2 hours, and subsequently plated on LB agar plates. Growth on the plates was examined following an overnight incubation at 52° C.

### DETAILED DESCRIPTION OF THE INVENTION

The present invention is directed to industrial fermentation processes for production of lactic acid from organic waste, in which a dried or semi-dried inoculum of Bacillus coagulans spores is used.

Organic waste management facilities handle collection, transport, processing, recycling/disposal and monitoring of waste materials. In order to recycle the waste into useful chemicals such as lactic acid, namely, utilize the organic waste as a substrate for industrial fermentation processes, an on-site fermentation system is typically required. The conventional method of inoculating industrial fermenters utilizes a wet inoculum of vegetative bacteria (wet seed train). This method has many disadvantages that makes it difficult to implement in waste management facilities, including the need to (i) tightly synchronize the wet seed preparation with the exact inoculation time of the production fermenter, (ii) have an on-site seed train production line which includes a few smaller scale fermenters for the production of the wet seed train (typically a ratio of 1:10 down to few liters flasks).

The wet seed train is a time-consuming and resource-exhausting process. It increases the production time, which consequently limits the number of fermentation cycles that can be performed per a given time period.

The present invention advantageously allows simple integration of lactic acid production into organic waste management facilities, for on-site production of lactic acid from the organic waste. The compositions of dried or semi-dried spores as disclosed herein can be easily transported to the waste management site, stored and removed from storage upon need.

In some embodiments, a need for seed line is eliminated by the present invention.

Using a dried or partially-dried spore inoculum have major advantages over the conventional wet inoculum, including: (i) avoiding the need to tightly synchronize the seed preparation with the inoculation time of the production fermenter; (ii) avoiding the need to have an on-site seed train production line which includes a few small scale fermenters for the production of a wet seed (typically a seed train of a ratio of 1:10 down to few liters flasks); (iii) an extended shelf-life, e.g. several months or longer, with a minimal effect on spores viability (in effect, a wet seed does not have any shelf-life); (iv) ease of transportation without special containers and conditions since the dried or partially-dried seed is much more resilient to uncontrolled transportation conditions; and (v) significantly reduced seed weight (e.g., over 95% weight reduction compared to a wet inoculum), due to water removal during its preparation, which significantly reduces transportation costs.

Importantly, the preparation of the dried or partially-dry seed can be done at a site that is separated by time and location from the waste management facility, thereby reducing the need for a skilled biotechnology engineer that is dedicated to prepare the seed at the waste management facility.

In addition, the fact that the dried or partially-dried seed can be prepared weeks or months ahead, put in storage and be available immediately for inoculating a production fermenter, significantly shortens the lactic acid production process.

Lactic acid production from organic waste typically comprises (i) degradation of polysaccharides that are present in the waste using one or more polysaccharide-degrading enzyme in order to release soluble reducing sugars that are suitable for fermentation (“saccharification”); and (ii) fermentation of reducing sugars to lactic acid by a lactic-acid producing microorganism (e.g., Bacillus coagulans as disclosed herein).

Renewable carbohydrate sources for lactic acid production typically include varied ratios of reducing sugars (glucose, fructose, lactose, etc.), but also large amounts of polysaccharides such as starch and optionally also lignocellulosic material. Typically, lactic acid-producing microorganisms can utilize reducing sugars like glucose and fructose, but do not have the ability to degrade polysaccharides like starch and cellulose. Thus, to utilize such polysaccharides the process requires adding polysaccharide-degrading enzymes, optionally in combination with chemical treatment, to degrade the polysaccharides and release reducing sugars. The integration of polysaccharide-degrading enzymes into the process may be sequentially, such that the substrate is treated with one or more polysaccharide-degrading enzymes and subsequently the lactic acid-producing microorganism is added and ferments the reducing sugars, or simultaneously, where the one or more polysaccharide-degrading enzymes and the lactic acid-producing microorganism are mixed together to perform simultaneous saccharification and fermentation. While the simultaneous process reduces the overall time that is required to obtain lactic acid from complex carbohydrate sources, one of its main challenges is the need to match the conditions for both bacterial growth and enzyme activity.

According to some embodiments, the methods of the present invention employ simultaneous saccharification and fermentation. Polysaccharide-degrading enzyme(s) are added to the organic waste together with a dried or partially-dried composition of Bacillus coagulans spores, to obtain simultaneous degradation of polysaccharides present in the waste and production of lactic acid.

When saccharification and fermentation are carried out as separate sequential steps, each step may take between about 18-24 hours. Conducting the two steps simultaneously significantly shortens the process, which results in improved productivity, as more organic waste can be converted to lactic acid per a given time period.

Bacillus coagulans Spore Compositions

Bacillus coagulans is a Gram-positive, thermophilic, facultative anaerobic, spore-forming bacterium that produces lactic acid, particularly L-lactic acid. B. coagulans has been proposed for industrial fermentation processes to produce L-lactic acid. B. coagulans has also been shown to maintain normal intestinal microflora and improve digestibility, and is commonly marketed as a probiotic to maintain the ecological balance of the intestinal microflora and normal gut function. For example, LactoSpore® is a Bacillus coagulans (MTCC 5856) spore preparation intended for use as a probiotic, containing a spray-dried powder of B. coagulans spores mixed with maltodextrin.

Yadav et al. (2009) Indian Journal of Chemical Technology, 16: 519-522 examined calcium lactate, calcium gluconate, Spirulina and maltodextrin as probiotic protectants of Bacillus coagulans during spray drying.

Bacillus coagulans strains that may be used according to the present invention include but are not limited to: B. coagulans ATCC 8038 DSM 2312, B. coagulans ATCC 23498 DSM 2314, B. coagulans MTCC 5856, B. coagulans PTA-6086 (GBI-30, 6086), B. coagulans SNZ 1969. Each possibility represents a separate embodiment of the present invention.

Spores may be prepared, for example, as follows: in the first step, a pure culture of B. coagulans is inoculated to a sterile seed medium and incubated on shaker at 50-55° C. for 12-24 hours. The seed culture is then transferred to a sporulation medium and incubated at 50-55° C. for 24-48 hours. Induction of sporulation requires stress conditions, for example, lack of nutrients, a relatively rich nitrogen source, such as yeast extract, along with limitation of the carbon and phosphor, presence of Mn²⁺ and Ca²⁺ ions, pH in the range of 5-6.5, incubation of 24-48 hours (preferably 24 hours), and combinations of the aforementioned stress-inducing factors. The spore concentration in the obtained spore culture is preferably at least 10\hat{ }7 spores/ml, more preferably at least 10\hat{ }8 spores/ml. Each possibility represents a separate embodiment.

Following incubation, the broth is harvested, centrifuged and the pellet is collected. In some embodiments, the harvested pellet, referred to herein as “semi-dried” or “partially-dried” preparation of the spores (moisture content in the range of 15%-30% w/w), is weighed and subsequently mixed with a magnesium lactate solution to obtain a composition comprising the harvested spores and 15-25% magnesium lactate (w/w of the total weight of the composition). In some embodiments, the concentration of magnesium lactate in the composition comprising the harvested spores (prior to drying) is in the range of 15-20% (w/w), for example, 15%, 16%, 17%, 18%, 19% or 20% (w/w) of the total weight of the composition. Each possibility represents a separate embodiment of the present invention. In some embodiments, the composition is dried, for example, spray-dried or heat-dried at 80° C., to obtain a dried spore composition in a powder form. The moisture content of a dried spore composition according to the present invention is up to 15% (w/w), preferably up to 10% (w/w), typically between 4% -10% w/w. Each possibility represents a separate embodiment of the present invention.

In some embodiments, heat selection at a temperature of 70° C.-80° C. is typically carried out following incubation and prior to drying.

In some embodiments, following drying, a dried composition in a powder form according to the present invention includes at least 10\hat{ }8 spores/g powder, for example, 10\hat{ }8-10\hat{ }10 spores/g powder. In some embodiments, a dried composition according to the present invention includes, for example 10\hat{ }8, 10\hat{ }9, 10\hat{ }10 spores/g powder. Each possibility represents a separate embodiment of the present invention. A dried composition according to the present invention further includes magnesium lactate, at a concentration of 40-60% (w/w), for example, 45%-55% (w/w), 40%-50% (w/w), 50%-60% (w/w). Each possibility represents a separate embodiment of the present invention.

In some embodiments, a dried composition of B. coagulans spores according to the present invention further comprises one or more polysaccharide-degrading enzyme selected from an amylase, a cellulase and a hemicellulase. In some particular embodiments, a dried composition of B. coagulans spores according to the present invention comprises a glucoamylase. In some exemplary embodiments, a dried composition of B. coagulans spores according to the present invention comprises a glucoamylase from Aspergillus niger.

In some embodiments, a dried composition according to the present invention does not require cold storage prior to use thereof. Thus, in some embodiments, a need for cold storage of the lactic-acid producing microbe is eliminated by the methods of the present invention.

According to the present invention, un-immobilized spores are used.

According to embodiments of the present invention, activation of the spores prior to inoculation into the fermenter is not required. For example, heat activation prior to inoculation into the fermenter is not required. As a further example, acid activation is not required prior to, or following, inoculation into the fermenter.

In some embodiments, following contacting with the organic waste substrate as disclosed herein, at least 90% of the spores germinate and produce vegetative cells, for example between 90%-100% of the spores germinate and produce vegetative cells.

Lactic Acid Production from Organic Waste

As used herein, the term “lactic acid” refers to the hydroxycarboxylic acid with the chemical formula CH₃CH(OH)CO₂H. The terms lactic acid or lactate (unprotonated lactic acid) can refer to the stereoisomers of lactic acid: L-lactic acid/L-lactate, D-lactic acid/D-lactate, or to a combination thereof.

For most industrial applications, L-lactic acid monomers with high purity (optical purity) are required in order to produce polylactic acid (PLA) with suitable properties. Thus, the methods and systems of the present invention are directed, in particular, to processes for the production of L-lactic acid or L-lactate salts at high yields.

Organic waste suitable for use according to the present invention is typically a complex organic waste comprising solid and non-solid materials. A complex organic waste includes carbohydrates for fermentation (soluble carbohydrates available for fermentation and/or polysaccharides that need to be decomposed via enzymes to release soluble carbohydrates for fermentation) and further contains impurities such as salts, lipids, proteins, color components, inert materials and more. Examples of organic wastes for use according to the present invention include, but is not limited to, food waste, organic fraction of municipal waste, agricultural waste, plant material, and a mixture or combination thereof. Each possibility represents a separate embodiment. Food waste in accordance with the present invention encompasses food waste of plant origin. Food waste in accordance with the present invention encompasses household food waste, commercial food waste, and industrial food waste. The organic food waste may originate from vegetable and fruit residues, plants, cooked food, protein residues, slaughter waste, and combinations thereof. Industrial organic food waste may include factory waste such as by products, factory rejects, market returns or trimmings of inedible food portions (such as peels). Commercial organic food waste may include waste from shopping malls, restaurants, supermarkets, etc. Plant material in accordance with the present invention encompasses agricultural waste and manmade products such as paper waste. Typically, organic waste comprises endogenous D-lactic acid, L-lactic acid or both L- and D- lactic acid, originating, for example, from natural fermentation processes, e.g., in dairy products.

Organic waste for use with the methods and systems of the present invention typically comprises complex polysaccharides including starch, cellulose, hemicellulose and combinations thereof. The organic waste also comprises soluble reducing sugars, and/or is saccharified with one or more polysaccharide-degrading enzyme to obtain soluble reducing sugars (fermentable carbohydrates). As used herein, the term “fermentable carbohydrates” refers to carbohydrates which can be fermented by Bacillus coagulans to lactic acid during a fermentation process. The reducing sugars typically comprise C5 sugars (pentoses), C6 sugars (hexoses) or a combination thereof. In some embodiments, said reducing sugars comprise glucose. In some embodiments, said reducing sugars comprise xylan.

Organic waste according to the present invention typically comprises complex polysaccharides and reducing sugars at varying ratios. The composition depends on the source of the waste, where some organic wastes may be more starch-rich (e.g., food waste from bakeries, mixed food waste of municipalities) and others may be rich with lignocellulosic material (e.g., agricultural waste). In some embodiments, the organic waste includes a combination of wastes from different sources.

In some embodiments, the percentage of at least one of starch, cellulose and hemicellulose in the organic waste is determined prior to treatment with one or more polysaccharide-degrading enzyme. In some embodiments, the percentage of soluble reducing sugars is determined prior to the fermentation.

Organic waste typically includes nitrogen sources and other nutrients needed for bacterial growth and lactic acid production, but such nutrients may also be supplied separately to the lactic acid production fermenter if needed.

Pretreatment of the organic waste according to the present invention typically includes decreasing particle size and increasing surface area, and also inactivating endogenous bacteria within the waste. In some embodiments, the pretreatment comprises shredding, mincing and sterilization.

Sterilization may be carried out by methods known in the art, including for example, high pressure steam, UV radiation or sonication.

The pretreatment may include, for example, shredding and sterilization. Pretreatment may also include mincing with an equal amount of water using a waste mincer, such as, e.g., an extruder, sonicator, shredder or blender.

In some embodiments, one or more saccharide-degrading enzyme and a dried or partially-dried composition of B. coagulans spores are added simultaneously to a fermentation reactor containing a pretreated organic waste. In additional embodiments, the time period between the addition of one or more saccharide-degrading enzyme and the addition of a dried or partially-dried composition of B. coagulans spores is in the range of 0-5 hours, including each value within the range. In other embodiments, one or more saccharide-degrading enzyme is added to the fermenter 1-5 hours after a dried or partially-dried composition of B. coagulans spores is added, for example, 1 hour, at least 2 hours, 2 hours, 3 hours, 4 hours or 5 hours after a dried or partially-dried composition of B. coagulans spores is added. Each possibility represents a separate embodiment. In other embodiments, one or more saccharide-degrading enzyme is added to the fermenter before a dried or partially-dried composition of B. coagulans spores is added.

As used herein, “mixing a dried composition of B. coagulans spores in a fermentation reactor”, “adding a dried composition of B. coagulans spores to fermentation reactor” and the like encompass adding the dried powder directly into the fermentation reactor, or reconstituting the powder in a reconstitution medium. The present invention particularly discloses reconstitution in a magnesium hydroxide slurry, to achieve both reconstitution and inhibition of microbial contaminants that may be present.

In some embodiments, a dried composition of B. coagulans spores is suspended in a magnesium hydroxide slurry prior to inoculation into the fermentation reactor. In additional embodiments, a dried composition of B. coagulans spores is suspended in a solution or slurry of other alkaline antimicrobial compounds prior to inoculation into the fermentation reactor, e.g., in a solution or slurry of an alkaline antimicrobial compound selected from the group consisting of magnesium oxide (MgO), calcium oxide (CaO), zinc oxide (ZnO) and calcium carbonate (CaCO₃). Each possibility represents a separate embodiment of the present invention.

Lactic acid fermentation according to the present invention is typically carried out under anaerobic or microaerophilic conditions, using batch, fed-batch, continuous or semi-continuous fermentation. Each possibility represents a separate embodiment of the present invention.

In batch fermentation, the carbon substrates and other components are loaded into the reactor, and, when the fermentation is completed, the product is collected. Except for an alkaline compound for pH control, other ingredients are not added to the reaction before it is completed. The fermentation is kept at substantially constant temperature and pH, where the pH is maintained by adding the alkaline compound.

In fed-batch fermentation, the substrate is fed continuously or sequentially to the reactor without the removal of fermentation broth (i.e., the product(s) remain in the reactor until the end of the run). Common feeding methods include intermittent, constant, pulse-feeding and exponential feeding.

In continuous fermentation, the substrate is added to the reactor continuously at a fixed rate, and the fermentation products are taken out continuously.

In semi-continuous processes, a portion of the culture is withdrawn at intervals and fresh medium is added to the system. Repeated fed-batch culture, which can be maintained indefinitely, is another name of the semi-continuous process.

Fermentations that produce acidic products such as organic acids etc. are typically performed in the presence of an alkaline compound, such as a metal oxide, a carbonate or a hydroxide. The alkaline compound is added to adjust the pH of the fermentation broth to a desired value, typically in the range of 4-7, including each value within the specified range. The alkaline compound further results in the neutralization of the L-lactic acid to a lactate salt. During fermentation the pH in the fermenter decreases due to the production of the lactic acid, which adversely affects the productivity of the Bacillus coagulans. Adding bases such as magnesium-hydroxide/oxide, sodium-hydroxide, potassium-hydroxide, or calcium-hydroxide adjusts the pH by neutralizing the lactic acid thereby resulting in the formation of a lactate salt.

In some particular embodiments, the present invention recycles organic waste to produce magnesium lactate. In some embodiments, such a process utilizes magnesium hydroxide as the alkaline compound for adjusting pH during fermentation. The fermentation results in lactate monomers and Mg²⁺ ions, that can be recovered as magnesium lactate.

Lactic acid fermentation is typically carried out for about 1-4 days or any amount therebetween, for example, 1-2 days, or 2-4 days, or 3-4 days, including each value within the specified ranges.

After fermentation is completed, the broth may be clarified by centrifugation or passed through a filter press to separate solid residue from the fermented liquid. The filtrate may be concentrated, e.g., using a rotary vacuum evaporator.

The fermentation broth according to the present invention may contain D-lactic acid originating from the organic waste. The D-LA is undesired in the production of L-LA for polymerization as it results in the formation of more D,D-lactide and meso-lactide, which adversely impact the quality of the PLLA final product. In some embodiments, the methods and systems of the present invention advantageously eliminate D-lactic acid by employing a D-lactic acid degrading enzyme or a D-lactic acid utilizing microorganism to the organic waste prior to lactic acid production, or to the fermentation broth during and/or following fermentation. Each possibility represents a separate embodiment.

Currently preferred is the use of a D-lactate oxidase as a D-lactic acid degrading enzyme. A D-lactate oxidase is an enzyme that catalyzes the oxidation of D-lactate to pyruvate and H₂O₂ using O₂ as an electron acceptor. The enzyme uses flavin adenine dinucleotide (FAD) as a co-factor for its catalytic activity. A D-lactate oxidase according to the present invention is typically a soluble D-lactate oxidase (rather than membrane-bound). Advantageously, the enzyme works directly in organic wastes and also in fermentation broths, to eliminate the D-lactic acid. In some embodiments, the D-lactate oxidase is from Gluconobacter sp. In some embodiments, the D-lactate oxidase is from Gluconobacter oxydans (see, for example, GenBank accession number: AAW61807). Elimination of D-lactate from fermentation broths derived from organic wastes using a D-lactate oxidase is described in WO 2020/208635 assigned to the Applicant of the present invention.

Suitable D-lactic acid-utilizing microorganisms within the scope of the present invention include, but are not limited to, an Escherichia coli lacking all three L-lactate dehydrogenases.

As used herein, “elimination”, when referring to D-lactic acid/D-lactate, refers to reduction to residual amounts such that there is no interference with downstream processes of producing L-lactic acid and subsequently polymerization to poly(L-lactic acid) that is suitable for industrial applications. “Residual amounts” indicates less than 1% (w/w) D-lactate, and even more preferably less than 0.5% (w/w) D-lactate, out of the total lactate (L+D) in a treated mixture of a fermentation broth at the end of fermentation. In some particular embodiments, elimination of D-lactate is reduction to less than 0.5% (w/w) D-lactic acid out of the total lactate in a fermentation broth at the end of fermentation.

According to further aspects and embodiments, L-lactate monomers are further purified. The L-lactate monomers may be purified as L-lactate salts. Alternatively, a reacidification step with, e.g., sulfuric acid, may be carried out in order to obtain crude L-lactic acid, followed by purification steps to obtain a purified L-lactic acid.

The purification processes may include distillation, extraction, electrodialysis, adsorption, ion-exchange, crystallization, and combinations of these methods. Several methods are reviewed, for example, in Ghaffar et al. (2014) Journal of Radiation Research and Applied Sciences, 7(2): 222-229); and López-Garzón et al. (2014) Biotechnol Adv., 32(5):873-904). Alternatively, recovery and conversion of lactic acid to lactide in a single step may be used (Dusselier et al. (2015) Science, 349(6243):78-80).

In some particular embodiments of the present invention, the alkaline compound used for pH adjustment during fermentation is magnesium hydroxide (Mg(OH)₂), resulting in a fermentation broth comprising lactate monomers and Mg²⁺, which can be recovered as magnesium lactate. A particular downstream purification process for purifying magnesium lactate via crystallization is described in WO 2020/110108, assigned to the Applicant of the present invention. The purification process can be applied to the fermentation broth after treatment that eliminates D-lactate monomers where applicable.

Saccharide-Degrading Enzymes

“Saccharide-degrading enzymes” as used herein refers to hydrolytic enzymes (or enzymatically-active portions thereof) that catalyze the breakdown of saccharides, including bi-saccharides (di-saccharides), oligosaccharides, polysaccharides and glycoconjugates. Saccharide-degrading enzymes may be selected from the group consisting of glycoside hydrolases, polysaccharide lyases and carbohydrate esterases. Each possibility represents a separate embodiment of the present invention. The saccharide-degrading enzymes for use with the present invention are selected from those that are active towards saccharides (such as polysaccharides) found in organic wastes, including food waste and plant material. In some embodiments, the saccharide-degrading enzymes may be modified enzymes (i.e., enzymes that have been modified and are different from their corresponding wild-type enzymes). In some embodiments, the modification may include one or more mutations that result in improved activity of the enzyme. In some embodiments, the saccharide-degrading enzymes are wild type (WT) enzymes.

The broad group of saccharide-degrading enzymes is divided into enzyme classes and further into enzyme families according to a standard classification system (Cantarel et al. 2009 Nucleic Acids Res 37: D233-238). An informative and updated classification of such enzymes is available on the Carbohydrate-Active Enzymes (CAZy) server (www.cazy.org).

In some embodiments, the saccharide-degrading enzymes used in the present invention are polysaccharide-degrading enzymes. In some embodiments, the polysaccharide-degrading enzymes are enzymes that degrade polysaccharides selected from starch and non-starch plant polysaccharides.

In some embodiments, the polysaccharide-degrading enzymes are glycoside hydrolases.

In some embodiments, the polysaccharide-degrading enzymes are selected from amylases, cellulases and hemicellulases. Each possibility represents a separate embodiment of the present invention.

A cellulase may be selected from, but not limited to: endo-(1 ,4)- -D-glucanase, εχo-(1,4)-β-\ddot{υ}-glucanase, β-glucosidases, Carboxymethylcellulase (CMCase); endoglucanase; cellobiohydrolase; avicelase, celludextrinase, cellulase A, cellulosin AP, alkali cellulase, and pancellase SS. Each possibility is a separate embodiment.

A hemicellulase may be a xylanase. Non-limiting examples of additional hemicellulases include arabinofuranosidases, acetyl esterases, mannanases, a-D-glucuronidases, β-xylosidases, β-mannosidases, β-glucosidases, acetyl-mannanesterases, a-galactosidases, -a-Larabinanases, and β-galactosidases. Each possibility represents a separate embodiment of the present invention.

An amylase may be selected from, but not limited to: glucoamylase, a-amylase; (1,4-a-D-glucan glucanohydrolase; glycogenase) β-Amylase; (1,4-a-D-glucan maltohydrolase; glycogenase; saccharogen amylase) γ-Amylase; (Glucan 1,4-a-glucosidase; amyloglucosidase; Exo-1,4-a-glucosidase; lysosomal a-glucosidase and 1,4-a-D-glucan glucohydrolase. Each possibility is a separate embodiment.

In some embodiments, the saccharide-degrading enzymes used in the present invention are disaccharide-degrading enzymes. In some embodiments, the disaccharide-degrading enzymes are selected from lactases and invertases. Each possibility represents a separate embodiment of the present invention.

The saccharide-degrading enzymes according to the present invention may be from a bacterial source. In some embodiments, the bacterial source is a thermophilic bacterium. The term “thermophilic bacterium” as used herein indicates a bacterium that thrives at temperatures higher than about 45° C., preferably above 50° C. Typically, thermophilic bacteria according to the present invention have optimum growth temperature of between about 45° C. to about 75° C., preferably about 50-70° C. Non-limiting examples of thermophilic bacterial sources for saccharide-degrading enzymes include: Cellulases and hemicellulases—Clostridium sp. (e.g. Clostridium thermocellum), Paenibacillus sp., Thermobifida fusca; Amylases—Bacillus sp. (e.g. Bacillus stearothermophilus), Geobacillus sp. (e.g. Geobacillus thermoleovorans), Chromohalobacter sp., Rhodothermus marinus. Each possibility is a separate embodiment.

In additional embodiments, the bacterial source of the saccharide-degrading enzymes is a mesophilic bacterium. The term “mesophilic bacterium” as used herein indicates a bacterium that thrives at temperatures between about 20° C. and 45° C. Non-limiting examples of mesophilic bacterial sources for saccharide-degrading enzymes include: Cellulases and hemicellulases—Klebsiella sp. (e.g. Klebsiella pneumonia), Cohnel sp., Streptomyces sp, Acetivibrio cellulolyticus, Ruminococcus albus; Amylases—Bacillus sp. (e.g. Bacillus amyloliquefaciens, Bacillus subtilis, Bacillus licheniformis), Lactobacillus fermentum. A person of skill in the art understands that some mesophilic bacteria (e.g., several Bacillus sp.) produce thermostable enzymes.

The saccharide-degrading enzymes according to the present invention may also be from a fungal source. Non-limiting examples of fungal sources for saccharide-degrading enzymes include: Cellulases and hemicellulases—Trichoderma reesei, Humicola insolens, Fusarium oxysporum; Amylases (e.g., glucoamylases)—Aspergillus niger Aspergillus oryzae, Penicillium fellutanum, Thermomyces lanuginosu.

Additional sources for saccharide-degrading enzymes for use in accordance with the present invention can be found, for example, at the CAZy server mentioned above.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### EXAMPLE 1

### Spore Preparation in Flasks

Bacillus coagulans was inoculated from a frozen stock into 5 ml LB (in 50 ml falcon). After overnight cultivation at 52° C., 200 rpm, 200 μl were added to 25 ml sporulation medium (0.4% yeast extract buffered with 40 mM potassium phosphate, pH 6.2). After 24 hours of cultivation (52° C., 200 rpm) a sample was taken for spore count and total count (vegetative cells and spores).

Counting was performed as follows: samples before and after heating (80° C. for 30 min) were serially diluted, plated on LB agar and counted. The plate count of non-heated samples represents total count of both vegetative cells and spores, while the plate count of heated samples represents only spore count (vegetative bacteria do not survive the high temperature).

The spore count reached ˜10\hat{ }7 spores/ml and equaled also the total bacteria count. Longer incubation times in the sporulation medium (up to 72 hours) had no effect on the spore count.

### EXAMPLE 2

### Spore Preparation in Fermenters

A. 6 ml of overnight B. coagulans grown in LB were inoculated into a 500 ml fermenter vessel containing 300 ml of a sporulation medium (0.4% yeast extract buffered with 40 mM potassium phosphate pH-6.2). pH was maintained at 6.5-7.0 using 10% phosphoric acid during spore fermentation. After overnight cultivation (52° C., 700 rpm, 0.3 VVM) a sample was taken for spore count and total count, as described above. The spore count reached ˜5*10\hat{ }6-10\hat{ }7 spores/ml, with the same total count, meaning that substantially all of the bacterial cells sporulated.

In a further experiment, a higher percentage of yeast extract (2.5%) was used in the sporulation medium. With pH control using 10% phosphoric acid and maintaining pH on 6.8, the spore count reached ˜10\hat{ }7 spores/ml.

B. 6 ml of overnight B. coagulans grown in LB were inoculated into a 500 ml fermenter vessel containing 300 ml of sporulation medium (0.4% yeast extract with 1% soybean peptone buffered with 40 mM potassium phosphate). pH-6.8 was maintained using 10% phosphoric acid. 48-72 hours growth (45° C., 400 rpm, 0.3 VVM) yielded ˜10\hat{ }8 spores/ml with same total count.

In a further experiment a higher percentage of yeast extract (2.5% yeast extract with 1% soybean peptone buffered with 40 mM potassium phosphate) was used, with no significant difference in spore yield (˜3*10\hat{ }8 spores/ml).

### EXAMPLE 3

### Lactic Acid Fermentation Using B. coagulans Spores—1^{st} Protocol

The following experiment tested the ability of B. coagulans spores to successfully germinate in organic waste (food waste) and produce lactic acid from sugars present in the waste. The experiment tested lactic acid production from organic food waste by inoculating B. coagulans spores and subsequently (3 hours later) adding a polysaccharide-degrading enzyme (a glucoamylase). In this setting, spore germination is induced by the temperature inside the fermenter (heat activation) and is supported by reducing sugars already found and available in the organic food waste prior to the addition of the polysaccharide-degrading enzyme.

The experiment was performed on organic food waste collected from supermarket rejects. The food waste was grinded and sterilized. Next, 300 mL of the pre-treated food waste were inoculated with 6*10\hat{ }4 spores/ml of B. coagulans spores (final concentration in the inoculated food waste) in a fermenter with a maximum working volume of 500 mL. The spores were kept in the medium in which they were prepared, in cold storage of 4° C., until use. After removal from storage the spores were immediately inoculated into the food waste.

The food waste inoculated with the spores was fermented at 52° C., pH 6.2. The pH was maintained using magnesium hydroxide. After a 3-hour incubation, 0.5 gr/L of a glucoamylase (GA) (Aspergillus niger) were added and the incubation continued under the same conditions of pH and temperature. Glucose and lactate concentrations were monitored during the process by using a RQflex10 (Merk) reader with appropriate sticks. Lactate synthesis began 4.5 hours after the addition of the spores. After only 22 hours, 95 gr/L lactate were measured. The glucose potential (the maximum amount of glucose that can be produced from the waste) was measured separately as a control and indicated 93 gr/L glucose potential. The results indicate substantially full conversion of the glucose to lactate, which indicates both good GA activity (saccharification) and good B. coagulans activity (spore initiation and lactate production) when inoculating the spores and 3 hours later adding the GA.

### EXAMPLE 4

### Lactic Acid Fermentation Using B. coagulans Spores—2^{nd} Protocol

The experiment was carried out on organic food waste collected from supermarket rejects. The food waste was grinded and sterilized. Next, 300 mL of the pre-treated food waste were inoculated with 7*10\hat{ }4 spores/ml of B. coagulans spores (final concentration in the inoculated food waste) and further mixed with 0.5 gr/L of a glucoamylase in a 500 mL fermenter. Fermentation was carried out at 52° C., pH 6.2. The pH was maintained using magnesium hydroxide. Glucose and lactate concentrations were monitored. Lactate synthesis began 4 hours after the addition of the spores. After a total of only 23 hours, 73 gr/L lactate were measured. The glucose potential (the maximum amount of glucose that can be produced from the waste) was measured separately as a control and indicated 72 gr/L glucose potential. The results indicate substantially full conversion of the glucose to lactate, which indicates both good GA activity (saccharification) and good B. coagulans activity (spore initiation and lactate production), when the GA and spores are mixed with the food waste simultaneously.

### EXAMPLE 5

### Preparation of a Dried Spore Formulation

A. B. coagulans spores (CFU 4.2*10\hat{ }7, sporulation medium: 0.4% yeast extract+40 mM potassium phosphate) were centrifuged at 13000 g for 30 minutes at 4° C. After supernatant removal, the pellet was weighed and subsequently resuspended in a magnesium lactate solution to obtain a formulation in which the magnesium lactate concentration is in the range of 15-25% (w/w) (% wt out of the total weight of the composition), for example 17% (w/w). The formulation was dried at 80° C. to obtain a dry powder and stored in a dark place at room temperature. The moisture content of the dried formulation with the magnesium lactate was in the range of 4%-10% w/w.

Samples were taken on Day 1 and Day 7 for spore count. For spore count, samples of the dry spore powder were resuspended in sterile tap water and mixed. Next, spore count was carried out as described in Example 1 by plate count of viable cells that germinate from the spores following plating on LB agar. The spore count reached 1.1*10\hat{ }7 spores/ml on Day 1 and 1.5*10\hat{ }7 spores/ml on Day 7. These results suggest that spore viability was maintained after the drying procedure and throughout storage at room temperature.

B. A suspension of B. coagulans spores in a sporulation medium (10\hat{ }8 spores/ml) was centrifuged at 13000 g to reduce volume by ×70 fold. The pellet was weighed and resuspended in a magnesium lactate solution to obtain a formulation in which the concentration of magnesium lactate is 17% (w/w, out of the total weight of the composition). The formulation was dried at 80° C. to obtain a dry powder of spores and magnesium lactate. The moisture content of the dried formulation was 9% (w/w). Spore concentration was 5*10\hat{ }10 spores/gr.

### EXAMPLE 6

### Preparation of a Semi-Dried Spore Formulation

B. coagulans spores (CFU 4.2*10\hat{ }7, sporulation medium: 0.4% yeast extract++40 mM potassium phosphate) were centrifuged at 13000 g for 30 minutes at 4° C. After supernatant removal, the pellet was weighed and subsequently resuspended in a magnesium lactate solution to obtain a composition in which the magnesium lactate concentration is in the range of 15-25% (w/w) (% wt out of the total weight of the composition), for example 17% (w/w). The moisture content of the semi-dried formulation with the magnesium lactate was ˜25% w/w, that is, in the range of 15% -30% w/w. The formulation was stored in a dark place at room temperature. Samples were taken on Day 1 and Day 7 for spore count as described above.

Spore count reached 2*10\hat{ }7 on Day 1 and 1.8*10\hat{ }7on Day 7. These results suggest that spore viability was maintained after the semi-drying procedure and throughout storage at room temperature.

### EXAMPLE 7

### Reconstitution of Dried Spore Formulations in a Magnesium Hydroxide Slurry

In the following experiments dried formulations of B. coagulans spores were suspended in 15% Mg(OH)₂ (w/w) aqueous slurry and subsequently incubated in the 15% Mg(OH)₂ slurry for varying incubation times. The slurry reaches a pH of >9.5. The effect on spore germination following the incubation and the ability of the slurry to prevent growth of microbial contaminants were examined.

A. B. coagulans spores in a dried form prepared as described in Example 5 were suspended in a 15% Mg(OH)₂ w/w aqueous slurry to obtain 10\hat{ }8 spores/ml. The suspension was aliquoted into four aliquots that were stirred at room temperature for 5, 30, 60 or 90 minutes. Next, samples were plated on LB agar plates and grown overnight at 52° C. Total bacteria count and spore count were performed after the overnight incubation as described in Example 1. The results are summarized in Table 1.

TABLE 1
Spore germination following incubation in 15% Mg(OH)₂

Bacteria count

| Incubation | Total bacteria | Spore count |
|---|---|---|
| time | count (bacteria/ml) | (spores/ml) |
| 5 min | 7*10\hat{ }8 | 1*10\hat{ }7 |
| 30 min | 2.6*10\hat{ }8 | 1*10\hat{ }7 |
| 60 min | 2*10\hat{ }8 | 1.1*10\hat{ }7 |
| 90 min | 2*10\hat{ }8 | 1.2*10\hat{ }7 |

The results showed that B. coagulans spores survive incubation in 15% Mg(OH)₂ and successfully germinate following such treatment. No differences were observed in the scale of bacterial cells that germinated from the spores between the different incubation times.

B. B. coagulans spores in a dried form prepared as described in Example 5 were suspended in a 15% Mg(OH)₂ w/w aqueous slurry to obtain 10\hat{ }8 spores/ml together with 5% of a glucoamylase dry powder. The suspension was aliquoted into five aliquots that were stirred at room_temperature for 5, 30, 60, 90 minutes, or 19 hours. Next, samples were plated on LB agar plates and grown_overnight at 52° C. The total bacteria count and spore count were performed after the overnight incubation as described in Example 1. The results are summarized in Table 2.

TABLE 2
Spore germination following incubation in 15% Mg(OH)₂

Bacteria count

| Incubation | | Total bacteria | Spore count |
|---|---|---|---|
| time | | count (bacteria/ml) | (spores/ml) |
| 5 | min | 2.6*10\hat{ }8 | 3.1*10\hat{ }7 |
| 30 | min | 2.7*10\hat{ }8 | 2.7*10\hat{ }6 |
| 60 | min | 2.9*10\hat{ }8 | 2.9*10\hat{ }6 |
| 90 | min | 2.5*10\hat{ }8 | 3.1*10\hat{ }6 |
| 19 | hours | 2.6*10\hat{ }8 | 1.3*10\hat{ }7 |

The results showed that B. coagulans spores survive incubation in 15% Mg(OH)₂ and successfully germinate following such treatment. No changes in the scale of bacterial cells that germinated from the spores were observed between the different incubation times. In addition, examination of the activity of the glucoamylase on starch following incubation in the 15% Mg(OH)₂ slurry for up to 90 minutes indicated that it remained active.

C. In order to examine the ability of the Mg(OH)₂ slurry to prevent growth of microbial contaminants and therefore provide aseptic conditions for inoculating the B. coagulans spores into lactic acid production fermenters, the following assay was carried out: Escherichia coli BL21, Bacillus subtilis strain 169 and Saccharomyces cerevisiae were added to 15% Mg(OH)₂ (10\hat{ }7 cells/ml) and incubated at 52° C. for 2 hours with shaking. A control sample was incubated in LB. Following incubation, the 15% Mg(OH)₂ mix and the LB mix were each plated on an LB agar plate and incubated at 52° C. to mimic fermentation conditions. The growth on the plates was examined following an overnight incubation

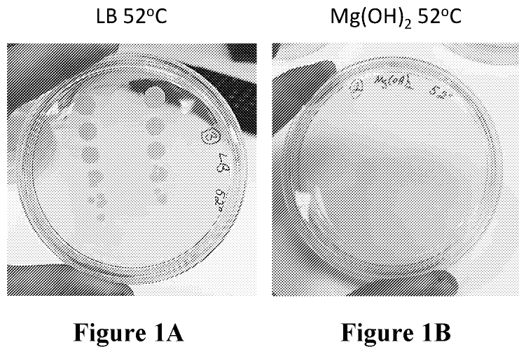

FIG. 1 shows that while in the control plate microbial colonies are clearly visible (indicating 8*10\hat{ }7 CFU), no growth is observed in the Mg(OH)₂ plate, indicating successful inhibition of microbial growth by incubation in 15% Mg(OH)₂.

### EXAMPLE 8

### Lactic Acid Fermentation Using Dried Formulations of B. coagulans Spores

A. Fresh B. coagulans spores and a dry formulation of B. coagulans spores (dried in 17% magnesium lactate, resuspended in sterile tap water) were used to ferment organic waste to lactate. Similar to the experiments described above, the fermentation was carried out on organic food waste collected from supermarket rejects, that was grinded and sterilized. Each inoculum was added to the fermenter in order to reach 5*10\hat{ }4 bacteria/ml. Glucoamylase was added to the fermenter together with the bacteria/spores. Fermentation was carried out at 52° C., pH 6.2. The pH was maintained using magnesium hydroxide. Glucose and lactate concentrations were monitored.

The results have shown substantially similar lag time (time between inoculation of the bacteria/spores and detection of lactate synthesis) and similar glucose conversion for both the fresh and dried inoculums: lag time was 4 hours for both fresh and dried inoculums, and glucose was fully converted to lactate regardless of inoculum type. The results therefore indicate that lactic acid production is not negatively affected by the use of a dried spore formulation to inoculate the fermentation.

B. B. coagulans spores in a dried form (dried in 17% magnesium lactate) were resuspended in sterile tap water or in a 15% Mg(OH)₂ slurry. 200 mg of the dry spore formulation were suspended in 2 mL of the respective liquid, mixed well by vortex, and added to a fermenter containing pretreated food waste (grinded and sterilized) in order to reach 1*10\hat{ }7 bacteria/ml. Glucoamylase was added to the fermenter together with the bacteria/spores. Fermentation was carried out at 52° C., pH 6.2. The pH was maintained using magnesium hydroxide. Glucose and lactate concentrations were monitored.

Lag time was 1.5 hour for the water-based inoculum and 3 hours for the Mg(OH)₂-based inoculum, however the overall process time was substantially similar for both inoculums, and glucose was fully converted to lactate regardless of inoculum type.

### EXAMPLE 9

### Exemplary Spore Concentrations in Various Formulae

1. Wet Formulations of Spores

1.1. Spore concentration in sporulation medium: at least 10\hat{ }8 spores/ml (=per gram)

1.2. Sporulation medium with 15%-25% w/w magnesium lactate: at least 10\hat{ }7 spores/ml (=per gram)

1.3. Sporulation medium with 15%-25% w/w calcium lactate: at least 10\hat{ }7 spores/ml (=per gram).

2. Semi-Dried Formulations of Spores (Following Centrifugation or Membrane Filtration)

2.1. Magnesium lactate: moisture content including capillary water is in the range of 20%-30% w/w (without drying)

2.2. Semi-dried formulation of spores with 15%-25% w/w magnesium lactate: at least 10\hat{ }8 spores/ml (=per gram)

2.3. Calcium lactate: moisture content including capillary water is in the range of 20%-30% w/w (without drying)

2.4. Semi-dried formulation of spores with 15%-25% calcium lactate: at least 10\hat{ }8 spores/ml (=per gram).

3. Dried Formulations of Spores (Following Heat Drying or Spray Draying)

3.1. Formulation with 15%-25% magnesium lactate: at least 10\hat{ }9 spores/gram

3.2. Formulation with 15%-25% calcium lactate: at least 10\hat{ }9 spores/gram.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-28. (canceled)

29. A method for recycling organic waste to produce lactic acid or a salt thereof, the method comprising:
providing a pretreated organic waste that was subjected to pretreatment comprising reduction of particle size and optionally sterilization;
(ii) providing a dried or partially-dried composition of Bacillus coagulans spores;
(iii) mixing the pretreated organic waste in a fermentation reactor with one or more saccharide-degrading enzyme and the dried composition of B. coagulans spores, and incubating the mixture in the fermentation reactor to saccharify the organic waste and induce germination of the spores and subsequently lactic acid production by vegetative B. coagulans cells that germinate from the spores; and
(iv) recovering lactic acid or a salt thereof from the fermentation broth.

30. The method of claim 29, further comprising suspending the dried composition of B. coagulans spores in a magnesium hydroxide slurry prior to the mixing with the pretreated organic waste in step (iii), thereby obtaining a B. coagulans spore suspension in which microbial contaminants are inactivated.

31. The method of claim 30, wherein the concentration of magnesium hydroxide in the slurry is in the range of 1%-25%.

32. The method of claim 30, wherein the suspending in a magnesium hydroxide slurry comprises incubating the suspension for 15-90 min at a temperature between 25-60° C.

33. The method of claim 29, wherein the dried composition of B. coagulans spores comprises magnesium lactate.

34. The method of claim 29, wherein the organic waste is selected from the group consisting of food waste, municipal waste, agricultural waste, plant material and a mixture or combination thereof.

35. The method of claim 29, wherein the incubating in step (iii) is carried out at a pH in the range of 5-7.

36. The method of claim 29, wherein the incubating in step (iii) is carried out at a temperature in the range of 45-60° C.

37. The method of claim 29, wherein the incubating in step (iii) is carried out for a period of time in the range of 20-48 hours.

38. The method of claim 29, wherein the one or more saccharide-degrading enzyme is a polysaccharide-degrading enzyme selected from the group consisting of an amylase, a cellulase and a hemicellulose.

39. The method of claim 29, wherein the one or more saccharide-degrading enzyme comprises a glucoamylase.

40. The method of claim 29, wherein the mixing in step (iii) comprises adding the dried composition of B. coagulans to the fermentation reactor to obtain at least 10\hat{ }4 spores/ml fermentation medium.

41. The method of claim 29, wherein the dried composition of B. coagulans spores is characterized by moisture content of less or equal to 10% w/w, or wherein the partially-dried composition of B. coagulans spores is characterized by moisture content in the range of 15%-30% (w/w).

42. A system for recycling organic waste to produce lactic acid or a salt thereof, the system comprising:
(a) a source of pretreated organic waste that was subjected to pretreatment comprising reduction of particle size and optionally sterilization;
(b) a dried composition of Bacillus coagulans spores;
(c) one or more saccharide-degrading enzyme; and
(d) a fermentation reactor for mixing therein the pretreated organic waste, the one or more saccharide-degrading enzyme and the dried composition of B. coagulans spores,
wherein the mixture is incubated in the fermentation reactor to saccharify the organic waste and induce germination of the spores and subsequently lactic acid production by vegetative B. coagulans cells that germinate from the spores.

43. The system of claim 42, wherein the system comprises:
(a) a source of pretreated organic waste that was subjected to pretreatment comprising reduction of particle size and optionally sterilization;
(b) a dried composition of B. coagulans spores suspended in a magnesium hydroxide slurry;
(c) one or more saccharide-degrading enzyme; and
(d) a fermentation reactor for mixing therein the pretreated organic waste, the one or more saccharide-degrading enzyme and the dried composition of B. coagulans spores suspended in a magnesium hydroxide slurry,
wherein the mixture is incubated in the fermentation reactor to saccharify the organic waste and induce germination of the spores and subsequently lactic acid production by vegetative B. coagulans cells that germinate from the spores.

44. A dried inoculum in a powder form for lactic acid fermentation, comprising spores of Bacillus coagulans and magnesium lactate, wherein the inoculum is dried and ready for inoculation into a lactic acid production fermenter to provide lactic acid production.

45. The dried inoculum of claim 44, wherein the dried inoculum comprises 10\hat{ }8-10\hat{ }10 spores/g powder, and wherein the concentration of the magnesium lactate in the dried inoculum is in the range of 40-60% (w/w).

46. A method for recycling organic waste to produce lactic acid or a salt thereof, the method comprising:
(i) providing the dried inoculum of claim 44, comprising B. coagulans spores and magnesium lactate;
(ii) suspending the dried inoculum in a magnesium hydroxide slurry, thereby obtaining a B. coagulans spore suspension in which microbial contaminants are inactivated;
(iii) mixing the suspension obtained in step (ii) in a fermentation reactor with one or more saccharide-degrading enzyme and pretreated organic waste that was subjected to pretreatment comprising reduction of particle size and optionally sterilization, and incubating to saccharify the organic waste and induce germination of the spores and subsequently lactic acid production by vegetative B. coagulans cells that germinate from the spores; and
(iv) recovering lactic acid or a salt thereof from the fermentation broth.

47. The method of claim 46, wherein the method is a method for producing magnesium lactate, comprising the following steps:
providing a pretreated organic waste that was subjected to pretreatment comprising reduction of particle size and optionally sterilization;
providing a dried inoculum in a powder form for lactic acid fermentation, comprising spores of Bacillus coagulans and magnesium lactate;
suspending the dried inoculum of B. coagulans spores in a magnesium hydroxide slurry, thereby obtaining a B. coagulans spore suspension in which microbial contaminants are inactivated;
mixing the pretreated organic waste in a fermentation reactor with one or more saccharide-degrading enzyme and the B. coagulans spore suspension;
incubating the mixture in the fermentation reactor to saccharify the organic waste and induce germination of the spores and subsequently lactic acid production by vegetative B. coagulans cells that germinate from the spores, wherein an alkaline compound selected from magnesium hydroxide, magnesium oxide and magnesium carbonate is added to the fermentation reactor during the incubation to adjust pH, thereby obtaining lactate monomers and Mg²⁺ ions; and
recovering magnesium lactate from the fermentation broth.

48. The method of claim 47, wherein the alkaline compound added to the fermentation reactor during the incubation to adjust pH is magnesium hydroxide.
